(19) United States
(12) Patent Application Publication
(10) Pub. No.: US 2025/0177449 A1
(43) Pub. Date: Jun. 5, 2025

(54) PREPARATION METHOD OF LYOPHILIZED PLATELET (LP) AND USE OF LP IN PREPARATION OF TRAUMATIC HEMOSTATIC PRODUCT

(71) Applicant: ARMY MEDICAL UNIVERSITY, PEOPLE'S LIBERATION ARMY, PRC, Chongqing (CN)

(72) Inventors: Zhaowen ZONG, Chongqing (CN); Chenglin DAI, Chongqing (CN); Lin CHEN, Chongqing (CN); Wenjuan DU, Chongqing (CN); Renqing JIANG, Chongqing (CN); Xin ZHONG, Chongqing (CN); Yijun JIA, Chongqing (CN); Can CHEN, Chongqing (CN); Haoyang YANG, Chongqing (CN); Song LIU, Chongqing (CN)

(21) Appl. No.: 19/050,030

(22) Filed: Feb. 10, 2025

(30) Foreign Application Priority Data
Jan. 8, 2025 (CN) 202510035666.9

(51) Int. Cl. A61K 35/19; A61K 9/08; A61K 31/7076; A61K 38/19; A61K 38/36; A61P 7/04; C12N 5/078

(52) U.S. Cl. CPC A61K 35/19; A61K 9/08; A61K 31/7076; A61K 38/19; A61K 38/36; A61P 7/04; C12N 5/0644

(57) ABSTRACT

A method for preparing lyophilized platelets and use in preparation of a traumatic hemostatic product are provided. The preparation method adopts appropriate lyophilization conditions, has a faster lyophilization process than conventional lyophilization processes, and can increase an activation rate of LPs to about 70% while maintaining an activity of the LPs. The lyophilized platelets prepared by the preparation method shows stronger migration ability than conventional LPs, have higher maximum amplitude (MA) of blood clots in in vitro and in vivo experiments than the conventional LPs, and exhibit an excellent hemostatic effect. In addition, the platelet transfusion stock solution has enhanced coagulation activity after transfusion into trauma simulation animals compared with the conventional LPs, and is distributed to injury sites in a larger quantity, thereby better promoting coagulation and improving post-traumatic coagulation dysfunction. Moreover, there are fewer platelets in the blood circulation, reducing a probability of ectopic thrombosis.

### CROSS-REFERENCE TO RELATED APPLICATION

This patent application claims the benefit and priority to the Chinese Patent Application No. 202510035666.9 filed with the China National Intellectual Property Administration on Jan. 8, 2025, the disclosure of which is incorporated by reference herein in its entirety as part of the present application.

### TECHNICAL FIELD

A preparation method of a lyophilized platelet (LP) and use of LP in preparation of a traumatic hemostatic product are provided, relating to the technical field of LPs.

### BACKGROUND

Platelets, an important component of blood, have one of their main functions being participating in thrombosis and coagulation. Platelet transfusions are necessary in the treatment of patients with massive blood loss and thrombopenia. Platelets are easily activated, instable, and have short lifespan, making them unfavorable for cryopreservation. Therefore, it is necessary to find a suitable storage method to extend the lifespan of platelets.

Lyophilization is a method of preserving cells and proteins by removing solvents such as water and then freezing. Lyophilized platelets (LPs) show stable performance at a room temperature, small size, and light weight, and are easy to store for a long time and convenient to transport. The LPs also demonstrate rapid clinical application, easy rehydration, and convenient pathogen sterilization.

LPs are initially used mainly for patients with thrombopenia due to various reasons. In the treatment of patients with thrombopenia, the longer the LPs circulate in vivo after platelet transfusion, the better the therapeutic effect will be, thereby allowing for a longer dosing interval. However, the situation is not exactly the same in cases of traumatic hemorrhage. When platelets are used to help control the hemorrhage, ideal LPs should have a same hemostatic function as the fresh platelets. After transfusion, such LPs can quickly distribute to the injured sites and play a role in promoting blood clot formation, but do not remain in vivo for too long to cause thrombosis in other parts of the body.

Some scholars believe that platelet activation inhibitors should be used before LPs transfusion to avoid platelet activation in order to maximize platelet function and increase a retention time of platelets in vivo after platelet transfusion. Currently, commonly used platelet activation inhibitors include amiloride, adenosine, sodium nitroprusside, and prostaglandin E1. These platelet function inhibitors all act on a cyclic nucleotide system of the platelets, stimulating the production of cyclic adenosine monophosphate (cAMP) and cyclic guanosine monophosphate (cGMP) to inhibit platelet activation. Prostaglandin El is widely used and has the effect of inhibiting platelet aggregation, thromboxane A production, atherosclerotic lipid plaque formation, and acting as immune complexes. Moreover, existing storage conditions are mostly only considered for the treatment of diseases such as thrombopenia. For example, a room temperature of 20° C. to 24° C. is beneficial for increasing the circulation time but negatively affects the coagulation effect. In patients with hemorrhage, the hemostatic effect is emphasized, while platelet circulation time is a relatively less important indicator since once platelets participate in coagulation, they will be quickly cleared out of the body.

### SUMMARY

In order to solve the above problems, the present disclosure provides a method for preparing a lyophilized platelet (LP) and use of LP in preparation of a traumatic hemostatic product. The LP prepared by the preparation method has a water content of 2% to 3.5%, an activation rate of about 70%, and migration ability stronger than conventional LPs. A maximum amplitude (MA) of blood clots detected by thromboelastography in in vitro and in vivo experiments is higher than that of conventional LPs, indicating a desirable hemostatic effect.

To achieve the above objective, the present disclosure provides the following technical solutions.

The present disclosure provides a method for preparing a lyophilized platelet (LP), including the following steps:
cooling a fresh platelet to −38° C. to −40° C. and holding for 60 min to obtain a first platelet;
subjecting the first platelet to vacuumization to 4.8 Pa to 5 Pa and cooling at −40° C. for 70 min to obtain a second platelet;
subjecting the second platelet to first heating to −28° C. to −30° C. and holding for 60 min to obtain a third platelet;
subjecting the third platelet to second heating to −18° C. to 20° C. and holding for 60 min to obtain a fourth platelet;
subjecting the fourth platelet to third heating to −10° C. and holding for 60 min to obtain a fifth platelet;
subjecting the fifth platelet to fourth heating to 0° C. and holding for 60 min to obtain a sixth platelet;
subjecting the sixth platelet to fifth heating to 10° C. and holding for 60 min to obtain a seventh platelet; and
subjecting the seventh platelet to sixth heating to 20° C. until the seventh platelet is dried to obtain the LP.

Preferably, the cooling is conducted at −2° C./min; the first heating is conducted at 0.2° C./min; the second heating is conducted at 0.2° C./min; the third heating is conducted at 0.2° C./min; the fourth heating is conducted at 0.2° C./min; the fifth heating is conducted at 0.2° C./min; and the sixth heating is conducted at 0.2° C./min.

Preferably, the LP has a water content of 2 wt. % to 3.5 wt. %.

The present disclosure further provides a platelet transfusion stock solution, including a platelet activator, a stromal cell-derived factor-1 (SDF-1), and an LP prepared by the preparation method.

Preferably, the platelet activator is one or more selected from the group consisting of thrombin, adenosine diphosphate (ADP), and ristocetin.

Preferably, the platelet transfusion stock solution includes the thrombin at a concentration of 1 U/mL, the ADP at a concentration of 200 μmol/L to 500 μmol/L, and the ristocetin at a concentration of 1.5 mg/mL to 2 mg/mL.

Preferably, the platelet transfusion stock solution includes SDF-1 at a concentration of 150 ng/mL.

Preferably, the platelet transfusion stock solution includes the LP at a concentration of (2−2.5)×10¹¹ platelets/L.

Preferably, the platelet transfusion stock solution includes a physiological saline as a solvent.

The present disclosure further provides use of the LP prepared by the preparation method or the platelet transfusion stock solution in preparation of a hemostatic product, where the hemostatic product includes a traumatic hemostatic product.

### Beneficial Effects

The preparation method adopts appropriate lyophilization conditions, has a faster lyophilization process than conventional lyophilization processes, and can increase an activation rate of LPs to about 70% while maintaining an activity of the LPs. The LP prepared by the preparation method shows stronger migration ability than the conventional LPs, has a higher MA of blood clots in in vitro and in vivo experiments than the conventional LPs, and exhibits an excellent hemostatic effect.

Moreover, the platelet transfusion stock solution provided by the present disclosure can promote the migration of transfused platelets to the bleeding sites through platelet activators and SDF-1 to increase the hemostatic effect, while reducing the incidence of thrombosis in unexpected sites. Experimental results show that the platelet transfusion stock solution has enhanced coagulation activity after transfusion into trauma simulation animals compared with the conventional LPs, and is distributed to injury sites in a larger quantity, thereby better promoting coagulation and improving post-traumatic coagulation dysfunction. Moreover, there are fewer platelets in the blood circulation, reducing a probability of ectopic thrombosis.

### BRIEF DESCRIPTION OF THE DRAWINGS

To illustrate the examples of the present disclosure or the technical solutions in the prior art more clearly, the accompanying drawings required in the examples will be briefly introduced below.

FIG. 1 shows the results of an adhesion experiment on different platelets;

FIG. 2 shows the results of a migration experiment on different platelets; where compared with Group A, * indicates P<0.05, ** indicates P<0.01; compared with Group C, ★ indicates P<0.05; compared with Group F, ▴ indicates P<0.05;

FIG. 3 shows the MA of blood clots with different platelets;

FIG. 4 shows the mean arterial pressures of rabbits in each group at different times;

FIG. 5A-FIG. 5B show the blood gas values of rabbits in each group at different times; where FIG. 5A represents a lactate value, and FIG. 5B represents a base excess value;

FIG. 6A-FIG. 6C show the changes in thromboelastogram (TEG) parameters of rabbits in each group at different times; where FIG. 6A represents an MA value,

FIG. 6B represents an R value, and FIG. 6C represents an α-angle value;

FIG. 7A-FIG. 7B show the coagulation values of rabbits in each group at different times; where FIG. 7A represents an APTT value, and FIG. 7B represents a PT value;

FIG. 8A-FIG. 8B show the platelet and hemoglobin counts of rabbits in each group at different times; where FIG. 8A represents a platelet count and FIG. 8B represents a hemoglobin count; and

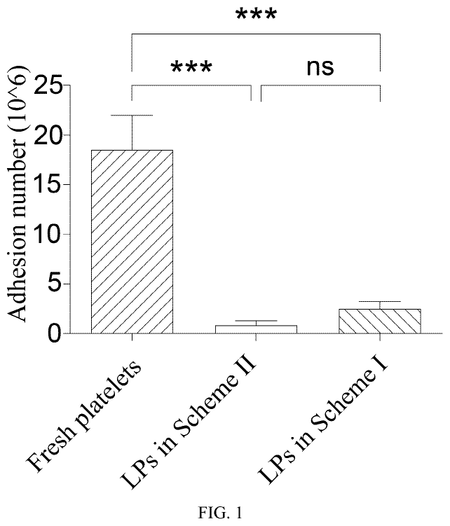

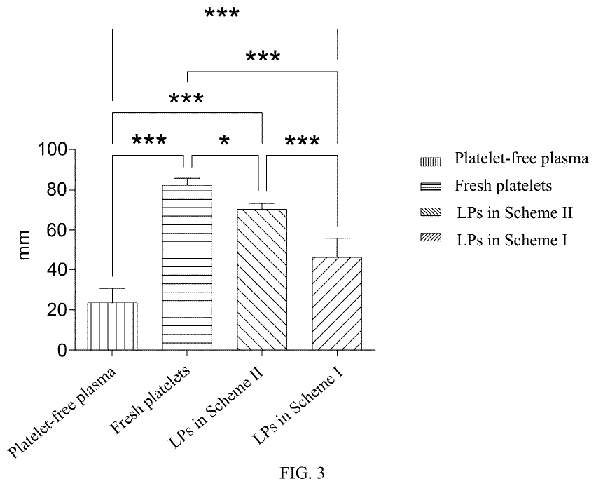

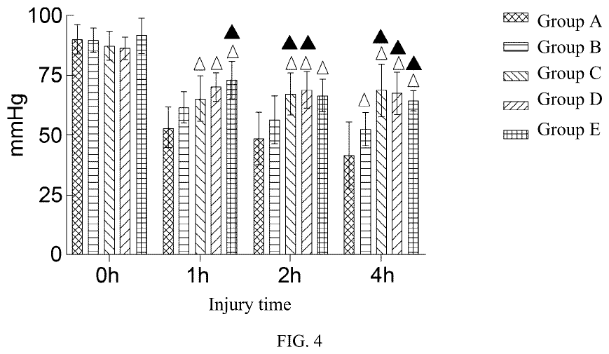

FIG. 9 shows the changes in blood urea nitrogen (BUN) index of rabbits in each group at different times; where
in FIG. 1 or FIG. 3, ns means P>0.05, * means P<0.05, *** means P<0.001; in FIG. 4 to FIG. 9, n=10, Δ: P<0.05 compared with Group A at the same time; ▴: P<0.05 compared with Group B at the same time, ♦: P<0.05 compared with the 0 h value of the same group; ⋄: P<0.05 compared with Group D at the same time.

### DETAILED DESCRIPTION OF THE EMBODIMENTS

The present disclosure provides a preparation method of a lyophilized platelet (LP), including the following steps:
cooling a fresh platelet to −38° C. to −40° C. and holding for 60 min to obtain a first platelet;
subjecting the first platelet to vacuumizing to 4.8 Pa to 5 Pa and cooling at −40° C. and holding for 70 min to obtain a second platelet;
subjecting the second platelet to first heating to −28° C. to −30° C. and holding for 60 min to obtain a third platelet;
subjecting the third platelet to second heating to −18° C. to −20° C. and holding for 60 min to obtain a fourth platelet;
subjecting the fourth platelet to third heating to −10° C. and holding for 60 min to obtain a fifth platelet;
subjecting the fifth platelet to fourth heating to 0° C. and holding for 60 min to obtain a sixth platelet;
subjecting the sixth platelet to fifth heating to 10° C. and holding for 60 min to obtain a seventh platelet; and
subjecting the seventh platelet to sixth heating to 20° C. until the seventh platelet is dried to obtain the LP.

In the present disclosure, a fresh platelet is cooled to −38° C. to −40° C. and held for 60 min to obtain a first platelet. As an embodiment, the fresh platelet is cooled to −40° C. and held for 60 min. As an embodiment, the cooling is conducted at −2° C./min.

In the present disclosure, the first platelet is subjected to vacuumizing to 4.8 Pa to 5 Pa and cooling at −40° C. and held for 70 min to obtain a second platelet. As an embodiment, the first platelet is subjected to vacuumizing to 5 Pa.

In the present disclosure, the second platelet is subjected to first heating to −28° C. to −30° C. and held for 60 min to obtain a third platelet. As an embodiment, the second platelet is subjected to the first heating to −30° C. and held for 60 min. As an embodiment, the first heating is conducted at 0.2° C./min.

In the present disclosure, the third platelet is subjected to second heating to −18° C. to −20° C. and held for 60 min to obtain a fourth platelet. As an embodiment, the third platelet is subjected to the second heating to −20° C. and held for 60 min. As an embodiment, the second heating is conducted at 0.2° C./min.

In the present disclosure, the fourth platelet is subjected to third heating to −10° C. and held for 60 min to obtain a fifth platelet. As an embodiment, the third heating is conducted at 0.2° C./min.

In the present disclosure, the fifth platelet is subjected to fourth heating to 0° C. and held for 60 min to obtain a sixth platelet. As an embodiment, the fourth heating is conducted at 0.2° C./min.

In the present disclosure, the six platelet is subjected to fifth heating to 10° C. and held for 60 min to obtain a seventh platelet. As an embodiment, the fifth heating is conducted at 0.2° C./min.

In the present disclosure, the seventh platelet is subjected to sixth heating to 20° C. until the seventh platelet is dried to obtain the LP. As an embodiment, the sixth heating is conducted at 0.2° C./min. As an embodiment, the LP has a water content of 2 wt. % to 3.5 wt. %.

The preparation method adopts appropriate lyophilization conditions, has a faster lyophilization process than conventional lyophilization processes, and can increase an activation rate of LPs to about 70% while maintaining an activity of the LPs. An LP prepared by the preparation method shows stronger migration ability than of the conventional LPs.

The present disclosure further provides a platelet transfusion stock solution, including a platelet activator, SDF-1, and an LP prepared by the preparation method. A problem that needs to be avoided when using the LP to treat bleeding is to prevent the formation of thrombi in non-injured sites of vital organs such as the brain and lungs. The platelet transfusion stock solution provided by the present disclosure can promote the migration of transfused platelets to the bleeding sites through platelet activators and SDF-1 to increase the hemostatic effect, while reducing the incidence of thrombosis in unexpected sites.

As an embodiment, the platelet activator is one or more selected from the group consisting of thrombin, ADP, and ristocetin. As another embodiment, the platelet activator is the thrombin.

As an embodiment, the platelet transfusion stock solution includes the thrombin at a concentration of 1 U/mL, the ADP at a concentration of 200 μmol/L to 500 μmol/L, and the ristocetin at a concentration of 1.5 mg/mL to 2 mg/mL.

As an embodiment, the platelet transfusion stock solution includes SDF-1 at a concentration of 150 ng/mL.

As an embodiment, the platelet transfusion stock solution includes the LP at a concentration of (2−2.5)×10¹¹ platelets/L.

As an embodiment, the platelet transfusion stock solution includes a physiological saline as a solvent.

The present disclosure further preferably provides a method for preparing the platelet transfusion stock solution, including the following steps:
dissolving the platelet activator and SDF-1 at a proportion to obtain a stimulant; and
mixing the stimulant and an LP prepared by the preparation method for 10 min to 30 min to obtain the platelet transfusion stock solution.

As an embodiment, the stimulant and the LP are at a volume-to-mass ratio of 1 mL:1 g.

As an embodiment, the platelet transfusion stock solution is diluted with a physiological saline before transfusion. Based on a mass of the LP, the platelet transfusion stock solution and the physiological saline are at a mass-to-volume ratio of 1 g:100 mL.

The present disclosure further provides use of the LP prepared by the preparation method or the platelet transfusion stock solution in preparation of a hemostatic product, where the hemostatic product includes a traumatic hemostatic product. As an embodiment, the traumatic hemostatic product may be a traumatic hemostatic product for traumatic hemorrhage, and applicable animal models thereof are a soft tissue injury combined with hemorrhagic shock model and a seawater immersion injury model. As an embodiment, a dosage form of the hemostatic product can be an injection.

In the present disclosure, the platelet transfusion stock solution has enhanced coagulation activity after transfusion into trauma simulation animals compared with the conventional LPs and is distributed to injury sites in a larger quantity, thereby better promoting coagulation and improving post-traumatic coagulation dysfunction. Moreover, there are fewer platelets in the blood circulation, reducing a probability of ectopic thrombosis.

The present disclosure further preferably provides a hemostasis method, including: transfusing an LP prepared by the preparation method or the platelet transfusion stock solution in combination with a lactated Ringer's solution. The LP combined with lactated Ringer's solution can significantly increase a mean arterial pressure of rabbits and alleviate acidosis, and the LP can continuously improve the coagulation after pre-activation.

In order to further illustrate the present disclosure, the preparation method of an LP and the use in preparation of a traumatic hemostatic product provided by the present disclosure are described in detail below with reference to the accompanying drawings and examples, but the accompanying drawings and the examples should not be construed as limiting the protection scope of the present disclosure.

### Example 1

1. Collection of platelets: after anesthesia, rabbits were cannulated through their femoral artery to collect whole blood, the whole blood was mixed with 40 g/L sodium citrate solution at a volume ratio of 9:1, centrifuged at 3,500 rpm for 20 min and then centrifuged for another 10 min until the plasma was clear, and then the plasma and the buffy coat were separated. After the buffy coat was converged, it was centrifuged again and the buffy coat was removed. 10 mL of lyophilized pretreatment solution was added to each 50 mL centrifuge tube, and the tube was shaken in a 37° C. shaking box for 1 h, and then centrifuged at 3,000 rpm for 5 min, and a resulting precipitate was removed, 10 mL of lyophilized buffer was added, and mixture in the tube was placed in a small bowl and spread flat, with the liquid surface thickness not exceeding 1 cm. The lyophilized pretreatment solution included the following components: 40 mmol/L trehalose, 100 mmol/L NaCl, 10 mmol/L KC1, 10 mmol/L EGTA, 10 mmol/L imidazole (pH=6.8), and 3% (v/v) DMSO; the lyophilized buffer included the following components: 9.5 mmol/L HEPEs, 142.5 mmol/L NaCl, 4.8 mmol/L KC1, 1.0 mmol/L MgCl2, 30 mmol/L trehalose, and 1% (v/v) bovine serum albumin, pH=6.8.

2. Preparation of LPs: the platelets collected in step 1 were frozen from 22° C. to −40° C. at −2° C./min for 60 min; after vacuumizing to 5 Pa, the platelets were maintained at −40° C. for 70 min, heated to −30° C. at 0.2° C./min for 60 min, heated to −20° C. at 0.2° C./min for 60 min, heated to −10° C. at 0.2° C./min for 60 min, heated to 0° C. at 0.2° C./min for 60 min, heated to 10° C. at 0.2° C./min for 60 min, and heated to 20° C. at 0.2° C./min and then dried (to a water content of 3 wt. %) to obtain the LPs.

### Example 2

A LP stimulant included the following components: 1 U/mL thrombin and 150 ng/ml stromal cell-derived factor-1 (SDF-1), with physiological saline as a solvent.

### Comparative Example 1

1. Collection of platelets: The method was the same as step 1 in Example 1.

2. Preparation of LPs: the platelets collected in step 1 were pre-frozen from room temperature to −45° C. at 20° C./h for 4 h; after vacuumizing to 5 Pa, the platelets were heated to −40° C. and held for 1 h, heated to −30° C. and held for 1 h, heated to −20° C. and held for 2 h, heated to −10° C. and held for 2 h, heated to 0° C. and held for 3h, heated to 10° C. and held for 3 h, heated to 20° C. and held for 4 h, and heated to 25° C. and then dried to obtain the LPs; where the heating rate after vacuumizing to 5Pa was 1° C./min.

### Test Example 1 In Vitro Experiment

### 1. Grouping and Stimulation of LPs

In the in vitro experiment, the lyophilized platelets were divided into 3 groups: Group A was fresh platelets, Group B was LPs prepared in Comparative Example 1(denoted as Scheme I), and Group C was pre-stimulated LPs. Specifically: 1 g of LPs prepared in Example 1 (denoted as Scheme II) and 1 mL of the LP stimulant in Example 2 were mixed for 30 min to obtain the pre-stimulated LPs.

### 2. Resuspension and Counting of LPs

The LPs or pre-stimulated LPs were dissolved and resuspended in platelet-poor plasma (PPP) at a ratio of 0.05 g of lyophilized powder added into 1 mL of PPP, and platelet counting was conducted using a hemocytometer.

### 3. Platelet Adhesion Assay

1 mg/mL collagen was added into an EP tube, and then added into a 96-well plate and incubated at 37° C. for 1 h. Excess collagen was washed away with PBS buffer, and 100 μL of platelets (fresh platelets or resuspended LPs) were added into each well and incubated at 37° C. for 2 h. After incubation, the suspension was removed by washing with PBS buffer, and 200 μL of 1% TritonX-100 was added into each well and lysed at 37° C. for 30 min. 120 μL of the supernatant was added into 60 μL of a detection working solution in the Beyotime LDH cytotoxicity detection kit, mixed well, incubated at room temperature in the dark for 30 min, and then the absorbance of each sample was measured using an enzyme reader. A series of known concentrations of platelet-rich plasma (PRP) were prepared, and the absorbance values were measured after lysis. The number of adhered platelets in the sample was calculated by making a standard curve based on the known platelet count and absorbance.

### 4. Platelet Migration Assay

The resuspended LP solution or fresh platelets were diluted with physiological saline to a concentration of (1−2)×10¹¹ platelets/L, and the platelet concentration of each group should be kept as close as possible. Platelets after different dilutions were divided into the following treatment groups: Group A (fresh platelets+SDF-1 stimulation), Group B (fresh platelets+no SDF-1 stimulation), Group C (pre-stimulated LPs+SDF-1 stimulation), Group D (pre-stimulated LPs+no SDF-1 stimulation), Group E (LPs prepared in Example 1+SDF-1 stimulation), and Group F (LPs prepared in Example 1+no SDF-1 stimulation). The group with SDF-1 stimulation was treated by adding SDF-1 to the diluted platelets to a final concentration of 150 ng/ml (simulating the up-regulation of local SDF-1 expression after in vivo trauma), while SDF-1 was added to the groups without SDF-1stimulation. The treatment methods of different groups were as follows:

A Transwell cell migration chamber with a pore size of 8 μm and a 6-well plate were prepared, 500 μL of physiological saline was added, incubated for 30 min to allow hydration, the liquid was removed, and whether there was liquid leakage was checked. 1.5 mL of PPP was added to a lower chamber of the migration chamber, and 1 mL of diluted platelets was taken from each treatment group and placed in the Transwell chamber; after preparation, the migration chamber and the cell culture plate were placed in a 37° C. incubator for 2 h, and the number of platelets in the lower chamber was detected while the migration rate of each group was calculated.

### 5. TEG of Platelet Suspension

The two batches of platelets from group B and group C were resuspended and then added with PRP and PPP samples to allow TEG detection. 1 mL of each sample was added to the activated coagulation reagent tube to allow reaction for 3 min, and then 20 μL of 0.1 M calcium chloride was added to the TEG sample cup. Subsequently, 340 μL of the sample was taken from the activated coagulation reagent tube and added into the test cup to measure TEG parameters such as coagulation reaction time (R value), blood clot MA, and blood clot formation rate (α-angle).

Experimental results were as follows:
1. The results of the platelet adhesion assay are shown in FIG. 1 and Table 1.

TABLE 1
Adhesion number of fresh platelets and LPs (n = 6)

| Groups | Group A | Group B | Group C |
|---|---|---|---|
| Adherent platelets (10⁶) | 18.56 ± 3.45 | 0.88 ± 0.27 | 2.46 ± 0.66 |

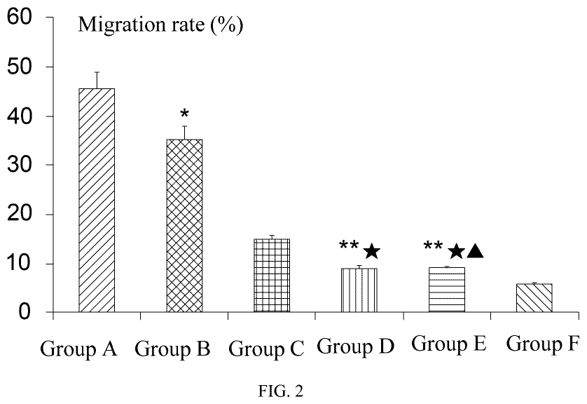

The results showed that compared with fresh platelets (Group A), the adhesion ability of LPs lyophilized by both methods was significantly decreased (P<0.001, FIG. 1). There was no significant difference in adhesion ability between Groups B and C (P>0.05, FIG. 1).
2. The results of the platelet migration assay are shown in FIG. 2. The results showed that the migration abilities of fresh platelets, LPs of Comparative Example 1, and LPs of Example 1 were significantly increased after stimulation with SDF-1. In comparison, the migration ability of the two groups of LPs after stimulation was significantly lower than that of fresh platelets after stimulation. Compared with conventional LPs (Comparative Example 1), the migration ability of LPs prepared in Example 1 was significantly enhanced after stimulation.
3. The MA values in TEG of platelet suspension are shown in FIG. 3 and Table 2.

TABLE 2
MA values of LPs, fresh platelets, and plasma-
deficient platelets in two groups (n = 6)

| | | | | Comparative |
|---|---|---|---|---|
| Item | PPP | PRP | Example 1 | Example 1 |
| MA/mm | 23.65 ± 6.96 | 82.08 ± 3.28 | 67.02 ± 7.47 | 46.65 ± 9.20 |

There was no significant difference in the α-angle and R value of platelets in each group, indicating that the lyophilization and pre-stimulation had no significant effect on the coagulation rate and the speed of coagulation initiation. The MA values reflecting platelet function showed differences (Table 1 and FIG. 2): the MA values of Groups B and C were significantly lower than those of Group A, while that of Group B was significantly lower than that of Group C. It was seen that pre-stimulation could enhance the coagulation activity of LPs, increase the MA, and thus improve the mass of blood clots and reduce bleeding.

### Test Example 1 In Vivo Experiment

1. Experimental animals and grouping design

The experimental animals used were 50 New Zealand white rabbits, provided by the Animal Experiment Center of Army Medical University, weighing (2.25+0.16) kg, aged 3 to 4 months, of either sex, and then the 50 rabbits were randomly divided into five groups: Group A, Group B, Group C, Group D, and Group E, with 10 rabbits in each group. The animal model used was a rabbit soft tissue injury combined with hemorrhagic shock and seawater immersion injury model established in the laboratory in an early stage. Specifically, the rabbits were anesthetized with 3% sodium pentobarbital at a dose of 1 mL/kg body weight, and then given soft tissue injury on left lower limb, while gauze was used to stop bleeding, and the blood loss of the soft tissue injury on lower limb was measured. The right lower limb was cannulated in the femoral artery, and then blood was drawn according to 26% of a total blood volume of the rabbit (the blood loss due to soft tissue injury on the lower limb was included), and then the rabbit was vertically immersed in 3% simulated seawater such that the seawater covered the xiphoid process, and then fished out after 15 min. A preparation method of the 3% simulated seawater included: artificial sea salt was added to tap water in proportion to prepare artificial simulated seawater with a salinity of 3%. The water temperature was adjusted to 22 to 23° C. by adding crushed ice within 1 h before the start of the experiment and during the experiment.

The established animal models were treated as follows according to the groups (kg in the following different groups referred to the weight of rabbits):
Group A: no resuscitation (i.e. no treatment);
Group B: rabbits were transfused with 20 mL/kg of lactated Ringer's solution;
Group C: rabbits were transfused with 20 ml/kg of lactated Ringer's solution and 3.78×10⁸ fresh platelets/kg;
Group D: rabbits were transfused with 20 mL/kg of lactated Ringer's solution and 3.78×108 LPs/kg; where the LPs were prepared in Example 1 and were rehydrated with physiological saline as a rehydration reagent, LPs: physiological saline =1 g: 100 mL;
Group E: rabbits were transfused with 20 mL/kg of lactated Ringer's solution and 3.78×108 pre-stimulated LPs/kg; a preparation method of the pre-stimulated LPs included: 1 g of LPs prepared in Example 1 was mixed with 1 mL of the LP stimulant in Example 2 for 10 min to obtain the pre-stimulated LPs; during the transfusion, physiological saline was added as a rehydration reagent for dilution, and the mass of LPs before pre-stimulation was calculated, pre-stimulated LPs: physiological saline=1 g: 100 mL.

The changes of injury condition in different groups were observed 4 h after the infusion for 20 min. The animals used in this experiment were approved by the Ethics Committee of the Army Medical University, and the experimental procedures complied with animal ethics requirements.

### 2. Detection Indicators and Detection Method

The mean arterial pressure (MAP) was monitored at four time points: before injury, and 1 h, 2 h, and 4 h after injury, and blood was drawn to test five coagulation items, blood routine examination, blood gas, 11 emergency items, and TEG. The number of red blood cells and platelets was measured using Mindray BC-5180CRP fully automatic blood cell analyzer (Mindray, Shenzhen, China). 1.5 mL of venous blood was collected at each time phase and centrifuged to measure prothrombin time (PT) and activated partial thromboplastin time (APTT) using a Rayto coagulation analyzer (Rayto, Shenzhen, China). The i-STAT handheld blood gas analyzer (Flextronics Manufacturing, Singapore) and its supporting CHEM8+ and CG4+ test cards were used to detect pH, lactate concentration (Lac), base excess (BE), and blood urea nitrogen (BUN) in four time phases. TEG analysis was conducted using a TEG instrument (CFMS LEPU-8800, Lepu Medical, Beijing, China). For each test, 1 mL of anticoagulated blood was drawn into the TEG activation coagulation reagent bottle to allow reaction for 3 min to 5 min. Subsequently, a common cup was installed and 20 μL of calcium chloride was added. After 3 min to 5 min, 340 μL of blood in the activation reagent bottle was drawn into the common cup and loaded for testing, and coagulation reaction time (R value), blood clot MA, and blood clot formation rate (α-angle) were recorded.

Experimental results were as follows:
1. The changes in MAP of rabbits in each group are shown in FIG. 4.

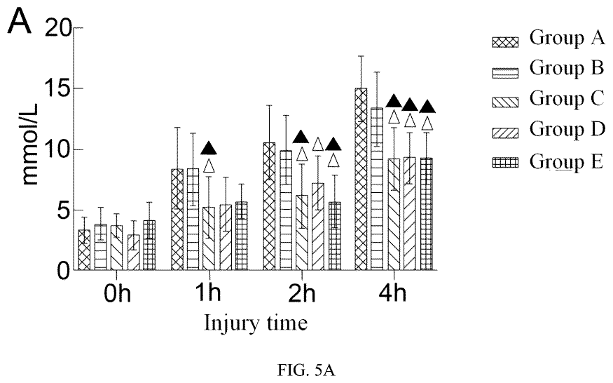

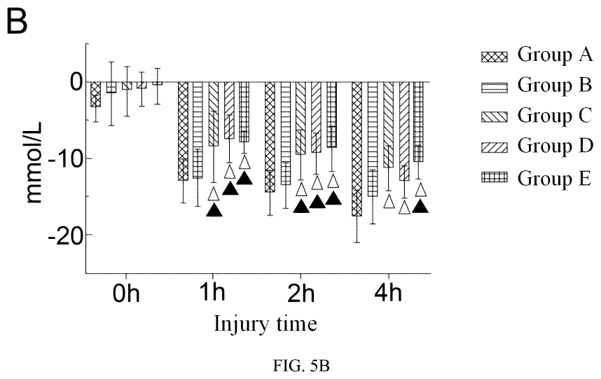

The results showed that there was no statistically significant difference in the baseline values of MAP in each group before injury. The MAPs in Groups C, D, and E were higher than that in Group A at 1 h after injury, and there was no significant difference in MAP between Groups A and B. At 2 h after injury, the MAPs in Groups C, D, and E were higher than that in Group A, and the MAPs in Groups C and D were higher than that in Group B. At 4 h after injury, the MAPs in Groups B, C, D, and E was higher than that in Group A, and the MAPs in Groups C, D, and E were higher than that in Group B.
2. The results of blood gas analysis of each group of rabbits after treatment are shown in FIG. 5A and FIG. 5B.

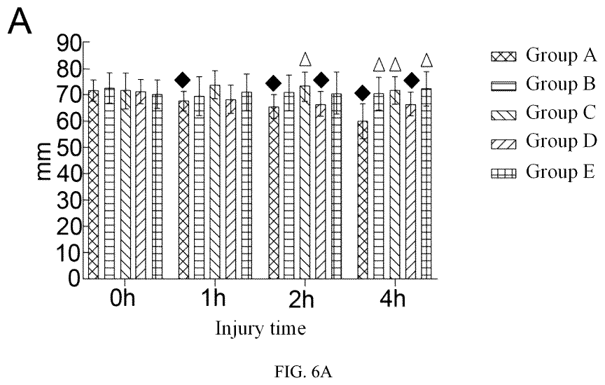

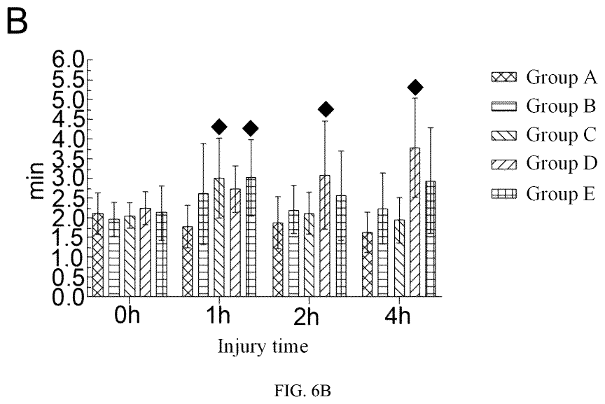

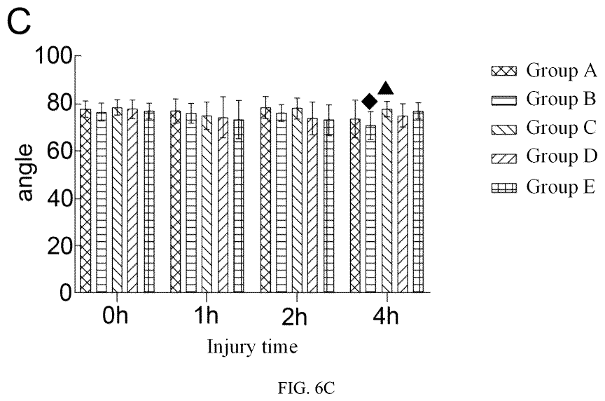

The results showed that there was no statistically significant difference in the baseline lactate values of the rabbits in each group before injury. At 1 h after injury, the lactate value of Group C was lower than that of Groups A and B. At 2 h after injury, the lactate levels in Groups C, D, and E were lower than those of Group A, and the lactate levels in Groups C and E were lower than those in Group B. At 4 h after injury, the lactate levels in Groups C, D, and E were all lower than those in Groups A and B (FIG. 5A). Base excess (BE) was another auxiliary indicator of acidosis. The results showed that at 1 h and 2 h after injury, the negative BE values in Groups C, D, and E were lower than those in Groups A and B. At 4 h after injury, the negative BE values in Groups C, D, and E were still lower than those in Group A, and the negative BE value in group E was lower than that in group B (FIG. 5B).
3. The TEG change trends of each group of rabbits after treatment are shown in FIG. 6A-FIG. 6C.

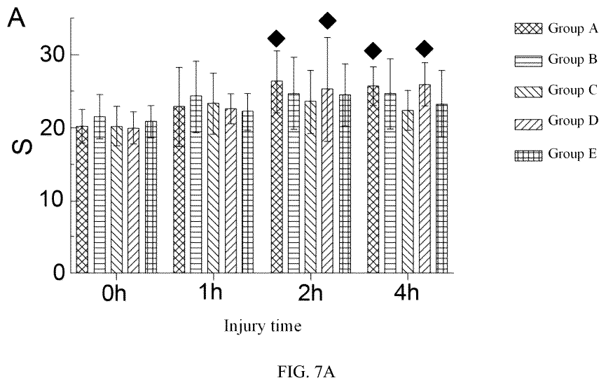

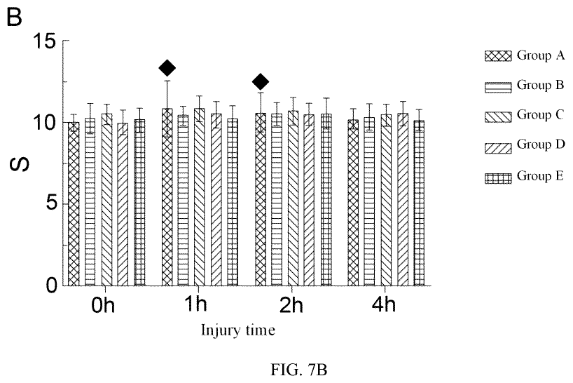

MA reflected platelet function. Among the 5 groups, the MA values of Groups A and D decreased significantly at 2 h and 4 h after injury compared with the initial time, while there was no significant difference in the MA values in the other three Groups B, C, and E between the initial time and the post-injury time. At 2 h, the MA value in Group C was significantly higher than that in Group A; at 4 h, the MA values in Groups B, C, and E were higher than those in Group A (FIG. 6A). The R value represented the coagulation reaction time and changed dynamically over time in each group. In group A, the R value showed a shortening trend, but it was not statistically significant. At 1 h after injury, the R values in Groups C and E showed a prolonging trend compared with their initial values. At 2 h and 4 h after injury, the R value in Group D also prolonged compared with the initial value (FIG. 6B). The α-angle value represented the rate of blood clot production, which tended to decrease over time in Groups A and B. At 4 h, the α-angle value in Group B was significantly lower than that at 0 h and lower that in group C, and the α-angle values in the other groups showed no significant differences at each time (FIG. 6C).
4. The changes in coagulation function of rabbits in each group after treatment are shown in FIG. 7A and FIG. 7B.

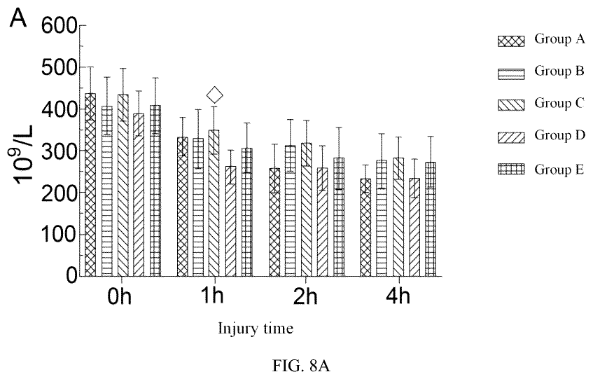

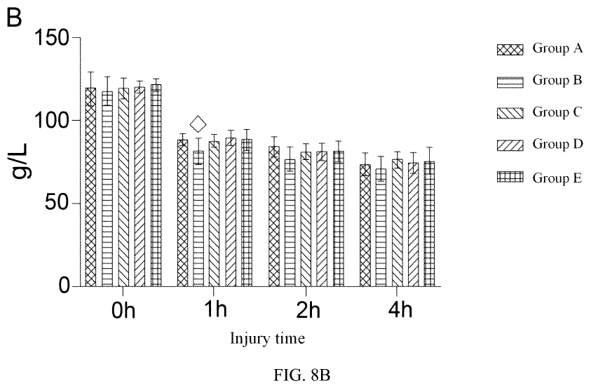

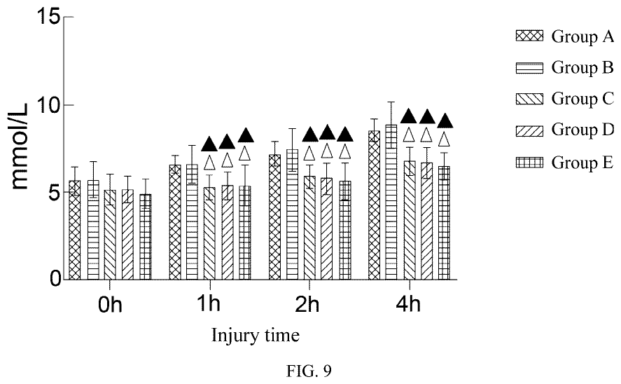

APTT reflected the endogenous coagulation status. In Groups A and D, the APTT at 2 h and 4 h after injury was prolonged compared with the initial value; the APTT in other groups showed a tendency to prolong after injury, but there was no significant statistical significance (FIG. 7A). PT reflected the exogenous coagulation status in vivo. The PT of Group A was prolonged at 1 h and 2 h after injury, while the other groups did not show obvious PT prolongation (FIG. 7B).
5. The changes in platelet and hemoglobin counts of rabbits in each group after treatment are shown in FIG. 8A and FIG. 8B.

The platelet counts of each group decreased significantly after injury, and the platelet count in Group C was higher than that in Group D at 2 h after injury. There were no significant differences in the platelet counts of each group at other time points (FIG. 8A). The hemoglobin (Hb) counts in each group also showed a downward trend after injury, and the Hb count in group B was lower than that in group D 2 h after injury (FIG. 8B).
6. The changes in BUN of rabbits in each group after treatment are shown in FIG. 9.

The BUN values of rabbits in each group increased to varying degrees at 4 h after injury. At the final 4 h, the BUN values in Groups C, D, and E were lower than that in Group A (FIG. 9).

In summary, in Group A without resuscitation after injury, MAP decreased to (53.12±8.38) mmHg, lactate increased, MA decreased, and APTT prolonged 1 h after injury (P<0.05). In comparison, the MAPs in Groups C, D, and E was higher than that in Group A at 1 h, 2 h, and 4 h after resuscitation (P<0.05). The lactate levels in Groups C, D, and E were lower than those in groups A and B at 4 h after injury (P<0.05). The MA values in Groups A and D at 4 h were significantly decreased compared with their initial values (P<0.05). The APTT and R values in Group D at 4 h were significantly prolonged compared with those at 0 h (P<0.05). The results indicated that LP combined with lactated Ringer's solution could significantly increase the MAP of rabbits and alleviate acidosis, and the LP can continuously improve the coagulation after pre-activation.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A method for preparing a lyophilized platelet (LP), comprising the following steps:
cooling a fresh platelet to −38° C. to −40° C. and holding for 60 min to obtain a first platelet;
subjecting the first platelet to vacuumizing to 4.8 Pa to 5 Pa and cooling at −40° C. and holding for 70 min to obtain a second platelet;
subjecting the second platelet to first heating to −28° C. to −30° C. and holding for 60 min to obtain a third platelet;
subjecting the third platelet to second heating to −18° C. to −20° C. and holding for 60 min to obtain a fourth platelet;
subjecting the fourth platelet to third heating to −10° C. and holding for 60 min to obtain a fifth platelet;
subjecting the fifth platelet to fourth heating to 0° C. and holding for 60 min to obtain a sixth platelet;
subjecting the sixth platelet to fifth heating to 10° C. and holding for 60 min to obtain a seventh platelet; and
subjecting the seventh platelet to sixth heating to 20° C. until the seventh platelet is dried to obtain the LP.

2. The method according to claim 1, wherein the cooling is conducted at −2° C./min; the first heating is conducted at 0.2° C./min; the second heating is conducted at 0.2° C./min; the third heating is conducted at 0.2° C./min; the fourth heating is conducted at 0.2° C./min; the fifth heating is conducted at 0.2° C./min; and the sixth heating is conducted at 0.2° C./min.

3. The method according to claim 1, wherein the LP has a water content of 2 wt. % to 3.5 wt. %.

4. A platelet transfusion stock solution, comprising a platelet activator, stromal cell-derived factor 1 (SDF-1), and an LP prepared by the preparation method according to claim 1.

5. The platelet transfusion stock solution according to claim 4, wherein the platelet activator is one or more selected from the group consisting of thrombin, adenosine diphosphate (ADP), and ristocetin.

6. The platelet transfusion stock solution according to claim 5, wherein the thrombin has a concentration of 1 U/mL, the ADP has a concentration of 200 μmol/L to 500 μmol/L, and the ristocetin has a concentration of 1.5 mg/mL to 2 mg/mL.

7. The platelet transfusion stock solution according to claim 4, wherein SDF-1 has a concentration of 150 ng/mL.

8. The platelet transfusion stock solution according to claim 4, wherein the LP has a concentration of (2−2.5)×10¹¹ platelets/L.

9. The platelet transfusion stock solution according to claim 4, physiological saline is used as a solvent.

10. The platelet transfusion stock solution according to claim 5, comprising physiological saline as a solvent.

11. The platelet transfusion stock solution according to claim 6, comprising physiological saline as a solvent.

12. The platelet transfusion stock solution according to claim 7, comprising physiological saline as a solvent.

13. The platelet transfusion stock solution according to claim 8, comprising physiological saline as a solvent.

14. A hemostatic product, comprising a traumatic hemostatic product, wherein the traumatic hemostatic product comprises the platelet transfusion stock solution according to claim 4.

15. The hemostatic product according to claim 14, wherein the platelet activator is one or more selected from the group consisting of thrombin, adenosine diphosphate (ADP), and ristocetin.

16. The hemostatic product according to claim 14, wherein the thrombin has a concentration of 1 U/mL, the ADP has a concentration of 200 μmol/L to 500 μmol/L, and the ristocetin has a concentration of 1.5 mg/mL to 2 mg/mL.

17. The hemostatic product according to claim 14, wherein SDF-1 has a concentration of 150 ng/mL.

18. The hemostatic product according to claim 14, wherein the LP has a concentration of (2−2.5)×10¹¹ platelets/L.

19. The hemostatic product according to claim 14, wherein physiological saline is used as a solvent.
